(19) United States
(12) Patent Application Publication
(10) Pub. No.: US 2021/0349077 A1
(43) Pub. Date: Nov. 11, 2021

(54) Method of quantifying induced membrane permeability and of screening compounds able to prevent said permeability

(71) Applicants: Dmitry Zaretsky, Chapel Hill, NC (US); Maria Zaretskaia, Chapel Hill, NC (US)

(72) Inventors: Dmitry Zaretsky, Chapel Hill, NC (US); Maria Zaretskaia, Chapel Hill, NC (US)

(73) Assignee: Zarbio, Chapel Hill, NC (US)

(21) Appl. No.: 17/302,647

(22) Filed: May 9, 2021

Related U.S. Application Data
(60) Provisional application No. 63/199,982, filed on Feb. 6, 2021.
(60) Provisional application No. 62/704,459, filed on May 11, 2020.

(51) Int. Cl. G01N 33/50; C12N 15/10; G01N 15/14

(52) U.S. Cl. CPC G01N 33/5041; G01N 15/1429; C12N 15/1093

(57) ABSTRACT

The present invention provides the method to quantify membrane permeability induced by various treatments including the formation of membrane pores/channels. Membrane channels created by misfolded (amyloidogenic) proteins are involved into development of various diseases, for which there is no known treatment, such as Alzheimer's disease, Amyotrophic Lateral Sclerosis, diabetes. The invention embodiments include methods to screen chemical entities for the ability to prevent increased membrane permeability. Finding chemical entities, which can prevent functioning of membrane channels formed by amyloidogenic peptides, is one of ways to develop treatments for said diseases. The invention embodiments can be used to observe the dynamics of formation of channels in biological or chemical systems where the channels are produced over time, for example to monitor channel formation by peptide fragments formed by proteases digesting full-length amyloidogenic peptides.

### TECHNICAL FIELD OF INVENTION

Present invention relates to the methods to measure permeability of lipid membranes (including cellular membranes) to various substances. The invented method can be used to estimate the effectiveness of treatments to prevent the increase in permeability caused by various means.

As an example of factor increasing permeability of lipid membranes are effects of so-called “misfolded” peptides and proteins. Such proteins usually do not have specific conformation immediately after synthesis and are soluble, but with time they form intra- and inter-molecular hydrogen bonds and create structures called beta-pleated sheets. These structures elongate into protofibrils, which can aggregate, become insoluble, and form clumps. Protein clumps formed in the biological tissues can be easily identified by histological staining. The diseases characterized by accumulation of such clumps are called “amyloid diseases”. The list of amyloid diseases includes but is not limited to Alzheimer's disease (AD), Parkinson's disease, Amyotrophic Lateral Sclerosis, Huntington's disease, diabetes mellitus type II, prion disease, Creutzfeldt-Jakob disease and many others.

It was demonstrated that before forming large insoluble protein clumps (with multiple molecules involved into single aggregate), at the stage of oligomers (only several molecules, as low as 3) these proteins can form a barrel-like structure which penetrates the lipid bilayer and forms membrane channel. This channel allows ions (such as Ca²⁺, Na⁺, K⁺), as well as organic molecules, and even macromolecules (such as dextrans) to go through the membrane. Essentially, if this process occurs in living cell, the cell loses the ability to control internal content. The first cells which are affected are excitable cells, which are dependent on the transmembrane ion gradients creating membrane potential.

There is a limited number of approaches to observe permeability of membranes. Two most wide-spread techniques are planar lipid bilayers (lipid bilayer is formed over small hole in a Teflon disc) or patch (membrane is formed in the opening of small glass pipette). Both approaches are labor-intensive, and difficult to apply to screening applications. Most importantly, each membrane serves as a single target, so it either allows observation of single channels (without an option to observe how many channels are formed) or measuring composite permeability (no way to distinguish few channels with high permeability vs many channels with low permeability). Similarly, in previous research disclosures when liposomes were used as a test object, it was impossible to distinguish few channels with high permeability vs many channels with low permeability.

Our invention creates an alternative way to observe channel formation—we can identify the number of channels formed in the suspension of liposomes, because each liposome is measured independently. Also, the technique allows for its use in high-throughput screening.

### BACKGROUND OF THE INVENTION

Diseases caused by misfolded proteins are very different. However, they have a common feature: immediately after the synthesis the protein has no secondary or tertiary structure and is soluble, but under various conditions it may undergo conformational changes, which ultimately result in the formation of beta-sheets and, after polymerization in the loss of the solubility. Individual molecules with intramolecular beta-sheet structure become linked to other such molecules forming oligomers, then elongate and form protofibrils. Protofibrils tend to aggregate and attract other molecules with relatively low solubility. As a result, insoluble conglomerates become large enough to be visible after histochemical staining of tissue sections. This was how these diseases were identified and grouped as amyloid diseases—various methods of staining reveal amorphous clumps of substance in brain or other tissues. Importantly, there was a correlation between where the clumps could be observed with clinical observations—dopaminergic areas contained such clumps in Parkinson's disease, while cortical areas are prone to the accumulation of clumps in Alzheimer's disease. Appearance of inclusions usually was accompanied by the disappearance of cells, such as dopaminergic neurons (Parkinson's disease) or cortical cells (Alzheimer's disease). Such correlation prompted early theory that the insoluble substance is the cause of the disease.

With time, the observations started to accumulate that clinical severity of disease does not necessarily is dependent on the number or the size of such inclusions. Importantly, the expression of inclusions has much better correlation with the length of disease than with the severity. Even more, the presence of inclusion does not necessarily result in the presence of the disease—there were multiple postmortem observations of highly expressed inclusions in medically healthy patients. However, there was strong correlation between the disappearance of neurons and clinical outcome. This led to the understanding that insoluble protein is a just another consequence of some process which is also responsible for cellular death.

Major promise to finding the cure for this group of diseases is in the comprehension of the process, which underlies the formation of insoluble protein inclusions, and the relationship of this process to the cellular death. Preventing cellular death is the only way to treat, delay the onset or slow down these diseases. Together with preventative screening and/or early diagnosis, such treatment can be a way to eradicate neurodegenerative diseases.

As it was mentioned above, freshly synthesized polypeptides do not have fixed conformation and are water-soluble. Over time, some molecules develop hydrogen bonds which fix specific turns and form beta-sheets, one of major secondary protein structures. Intramolecular hydrogen bonds fix turns within the molecules (label 1 at the FIG. 1), while intermolecular bonds attach multiple polypeptide molecules to each other (label 2 at the FIG. 1) forming oligomers (label 3 at the FIG. 1). Structure-wise, protofibrils are formed by core pleated beta-sheet structures with short peptide tails spreading to the sides of the core. Interaction between protofibrils through peptide tails results in the formation of large fibrils, the process which may also include other proteins (label 5 at the FIG. 1) which become stuck on the protofibrils and remain trapped in the insoluble protein clumps.

It is now become wide-accepted that cellular or neuronal toxicity is mediated by oligomeric structures, while soluble monomers and formed insoluble large-size fibrils appear mostly non-toxic. The mechanism of cellular toxicity induced by oligomers is intensely studied. Multiple pathways were proposed from increased lipid peroxidation to the release of cytokines by immune-competent cells. Among feature which is characteristic for all studied peptides known to be involved in amyloid diseases is that they affect intracellular electrolyte balance including the increase of intracellular calcium. It was demonstrated that the mechanism involves the formation of protein channels in cell membranes after physical interaction of polypeptide with said membranes. The size of oligomers which are most toxic to cells is estimated to be in low single digit numbers, such as trimers (three molecules per globule which is binding the cell).

To treat the AD, we need to prevent or slow down the processes which ultimately result in neuronal death. Importantly, the very process which initiates cellular toxicity, the insertion of amyloid into the cellular membrane and functioning of ion channels, is not targeted by currently available drugs. We are strongly convinced that the major reason for the absence of such treatments is the absence of techniques which allow high throughput studies of ion disturbances induced by misfolding peptides in general, and by amyloid peptides in particular. In this invention, we claim that proposed technique can be used to study the formation of channels in artificial and cellular membranes, and that this technique can be used to screen chemical entities able to prevent ion disturbances induced by channel-forming peptides.

### SUMMARY OF INVENTION

In this invention we describe the method to identify the formation of functional ion channels in model lipid membranes and the method of high throughput screening of substances which are able to prevent disturbances induced by membrane channel.

Various embodiments of present invention provide the methods of high-throughput testing of peptides which are able to form membrane channels; ways to identify permeability characteristics of membrane channels, as well as methods of screening compounds for potential medical use to treat diseases which are developing due to misfolding of proteins and formation of membrane ion channels.

### BRIEF DESCRIPTION OF DRAWINGS

FIG. 1. The schematic of polymerization of amyloidogenic proteins.

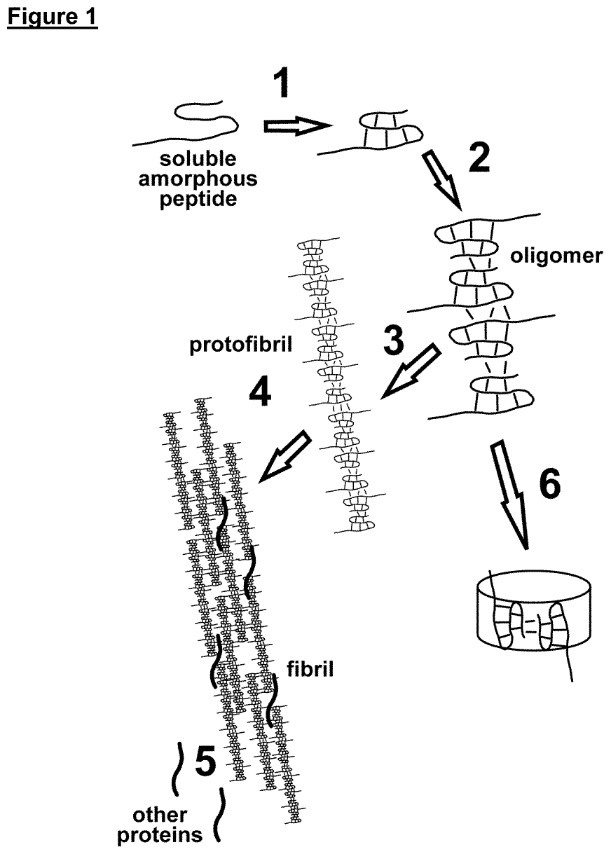

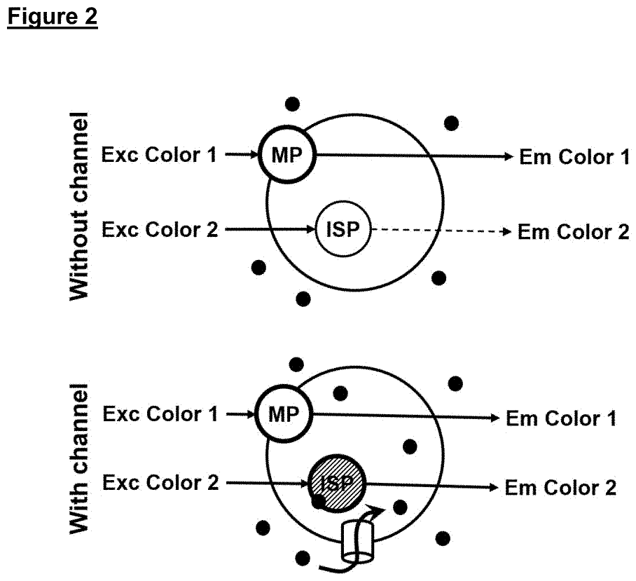

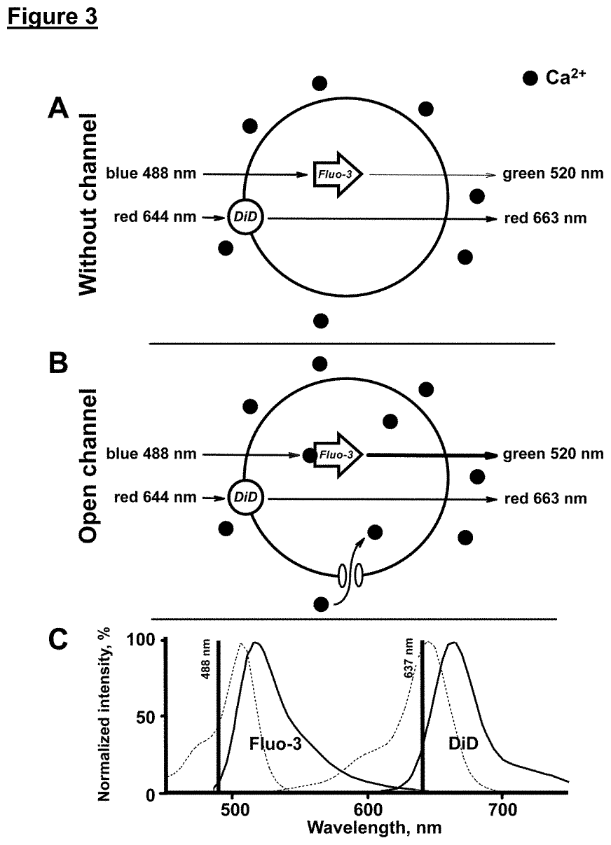

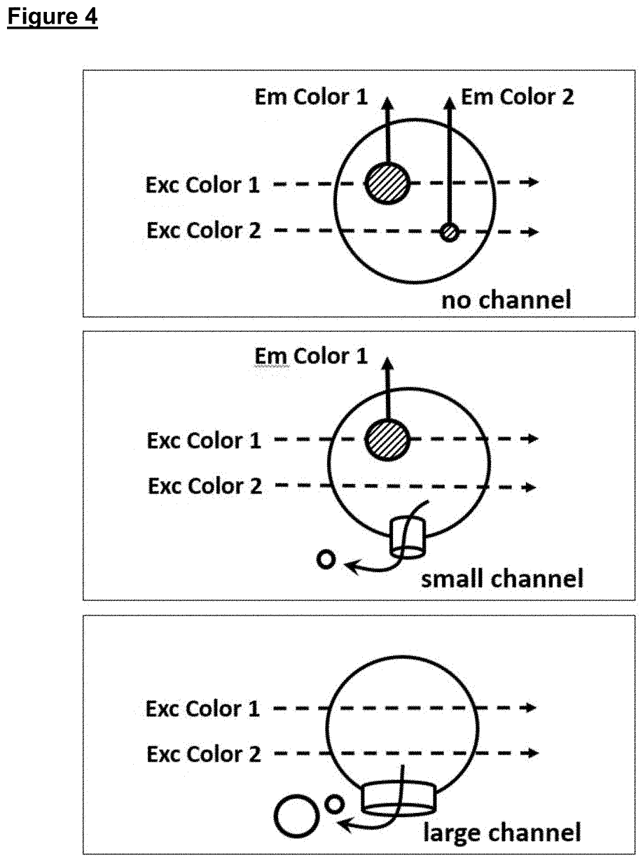

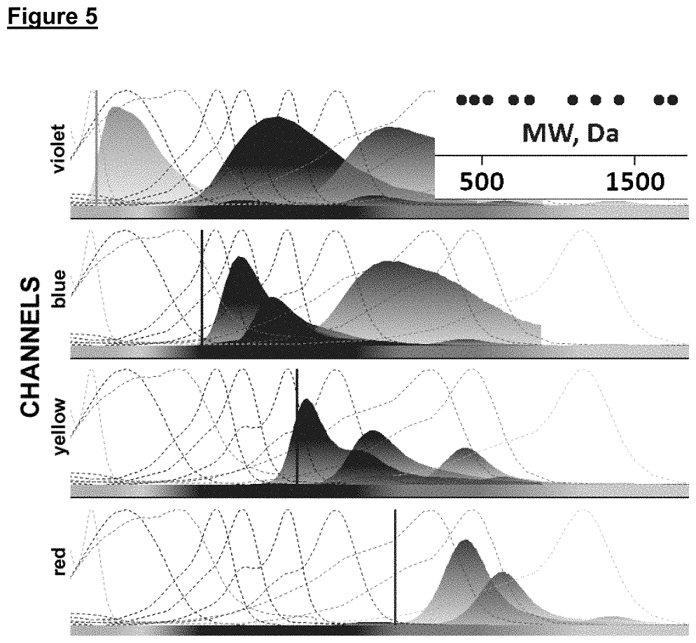

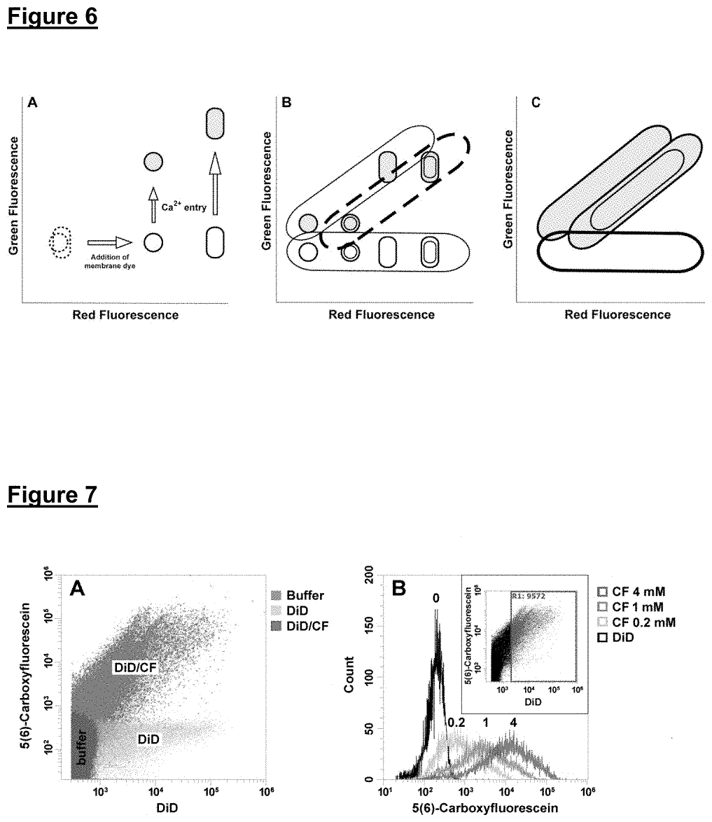

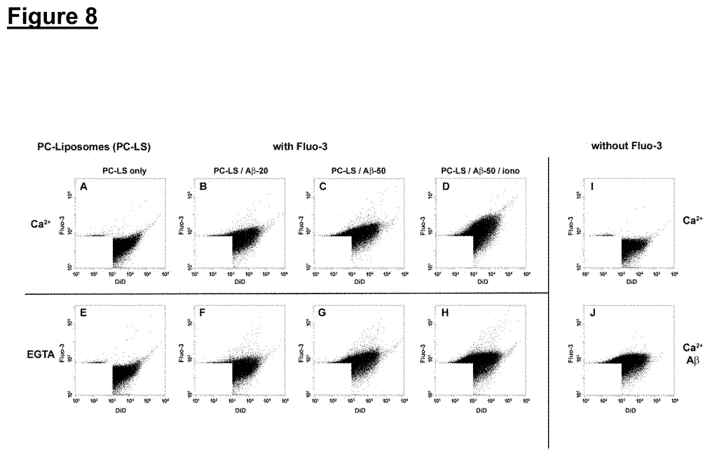

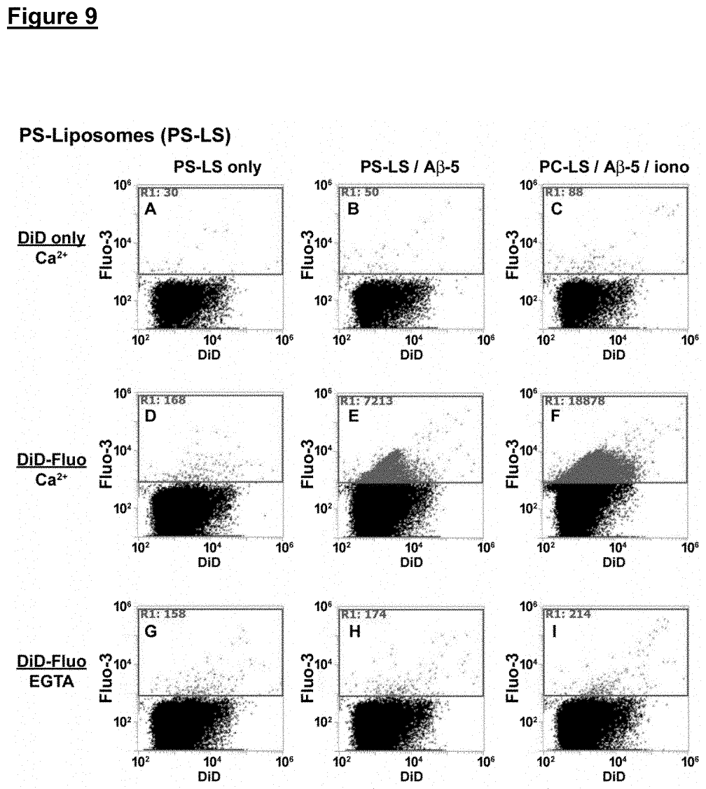

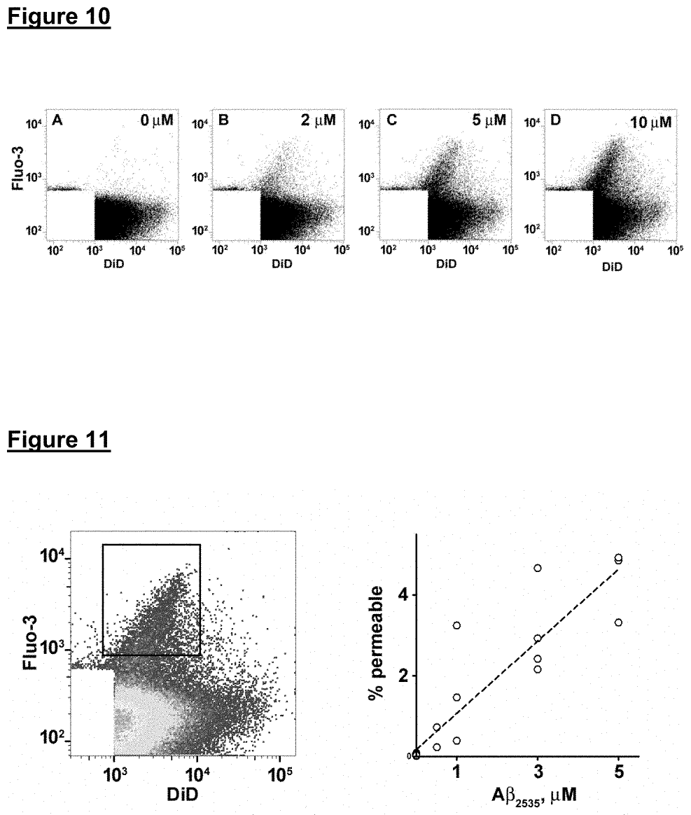

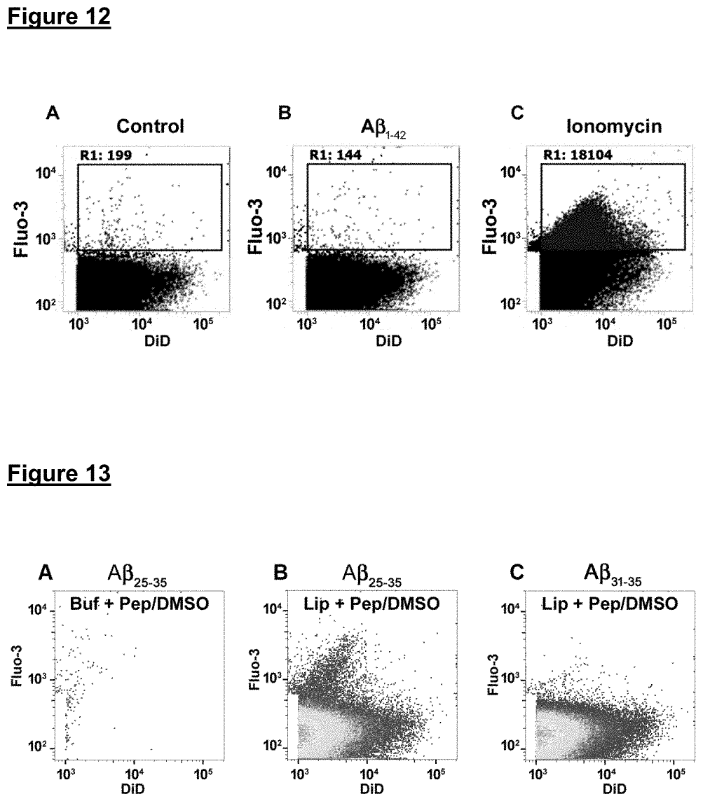

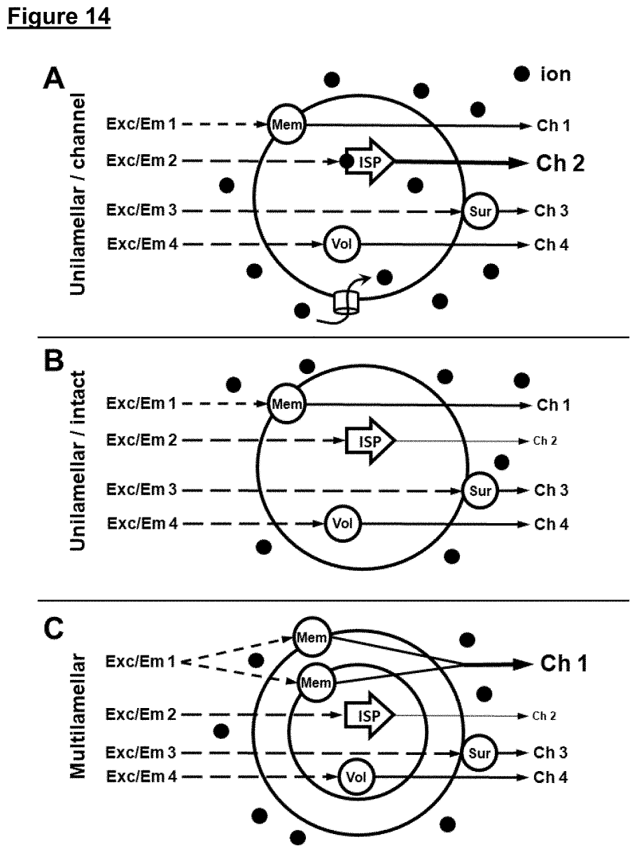

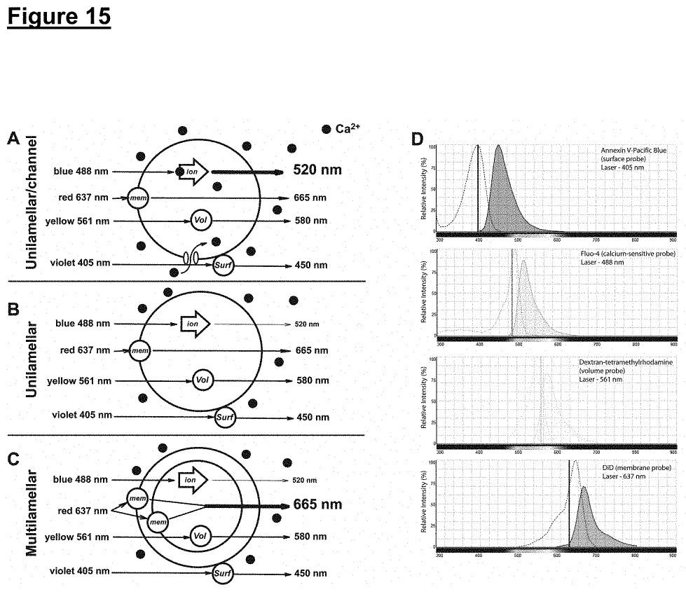

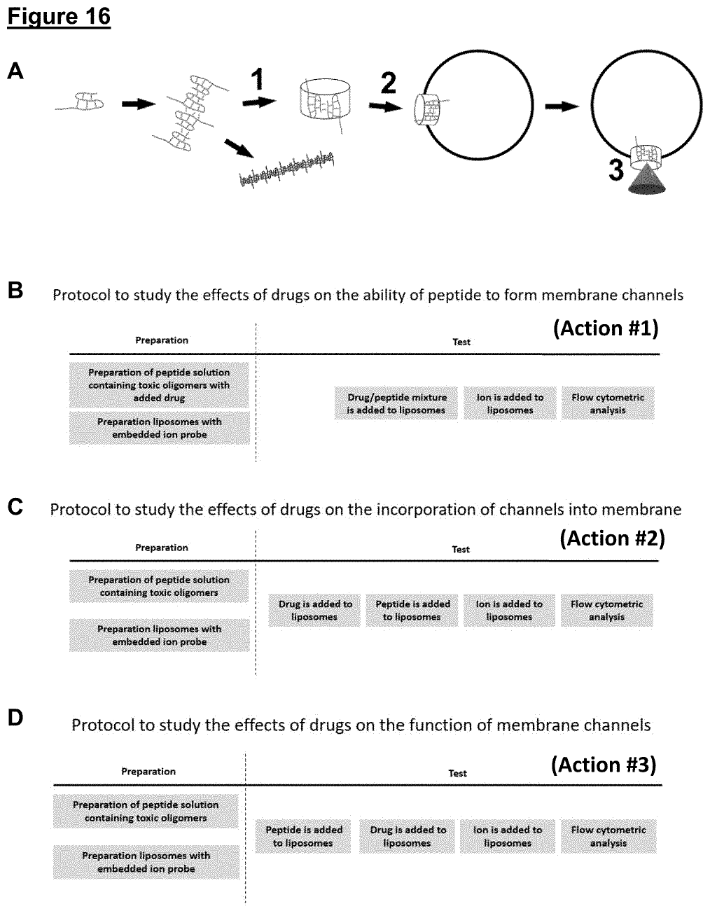

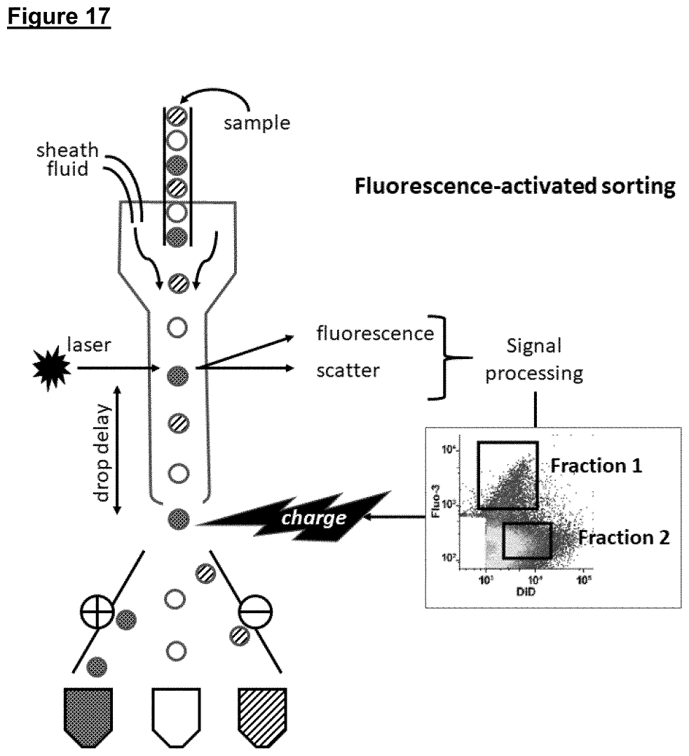

Amyloid peptides are initially soluble without secondary or tertiary structure. With time, they are stabilized by intra- and intermolecular hydrogen bonds (1 and 2, correspondingly) forming beta-pleated sheets (one of major secondary structures in proteins). Elongation of these supramolecular structures results in formation of protofibrils which have (3-sheet core with polypeptide tails looking to the sides of the protofibril (3). Protofibrils stick to each other through interaction between side polypeptide chains (4) and may involve other proteins (5), which may or may not be containing carbohydrate and lipid components (glyco- and lipoproteins). At oligomeric stage, beta-sheet can form barrel-like structures (6), which can incorporate into lipid membranes and serve as ion channels.

FIG. 2. The schematic of simplest generic fluorescence setup for high-throughput testing of the ion channel formation in artificial membranes (liposomes).

The formation of ion channels is best observed in unilamellar liposomes because the channels are not formed only in the outer membranes, but not in the internal membranes; therefore, in multilamellar liposomes, ions can not reach the internal volume of the vesicle. Usually, preparatory techniques to form unilamellar liposomes result in the suspension of vesicles with the diameters less than one micrometer, so they do not effectively scatter light. In case, we are interested to estimate the number of formed channels, it is possible to count the number of permeabilized vesicles, for example, using a method of flow cytometry. To identify objects which are smaller than the wavelength in the flow, it is required to use the parameter other than scattering, which is usually used to identify cells that have the size of several micrometers or more. To identify liposomes in the flow, one of possibilities is to add lipid-soluble fluorescent probe (MP, membrane probe), so the vesicle can be identified using intrinsic fluorescence.

The liposomes are prepared in the solution containing ion-sensitive fluorescent probe (ISP) in ion-free medium and are cleared from extravesicular ISP. Membranes are impermeant to the ion, so even after the addition of ion to the medium, ISP remains free of calcium and has typical calcium-free fluorescent properties. If the membrane become permeant to calcium, for example because of channel formation, ions enter the liposomes, bind ISP, so ISP fluorescence has ion-bound properties (either the intensity changes dramatically, or spectra of excitation and/or emission shift in ratiometric probes). The figure represents the situation when the intensity of fluorescence of ion-bound probe increases after binding ion, so impermeable liposomes are non-fluorescent in the measurement channel corresponding to the ISP, while permeable liposomes are intensely fluorescent in the same channel.

Probes are selected in a way that allows for reliable measuring specific fluorescence of each probe through selection of excitation and emission wavelengths (colors)—“Exc Color” and “Em Color”, correspondingly.

FIG. 3. The possible implementation of fluorescence setup for testing of the calcium channel formation in liposomes.

Unilamellar liposomes are created with Fluo-3 membrane-impermeant fluorescent dye sensitive to the concentration of calcium (an example of ion sensitive probe, ISP, at the FIG. 2). The buffer for the created liposomes is calcium-free, and can contain calcium-chelating agents, such as EGTA or EDTA. Both calcium-free and calcium-bound Fluo-3 effectively absorb light with wavelength of 488 nm (one of typical lasers used in flow cytometers, blue light). However, calcium-free Fluo-3 does not emit fluorescence, while calcium-bound Fluo-3 emits light with maximum around 520 nm (green). Measuring the fluorescence at 520 nm allows to distinguish the liposomes which contain free calcium and those which are impermeant to calcium.

To identify liposomes in the flow, lipids can be supplemented with a lipophilic dye such as 1,1′-dioctadecyl-3,3,3′,3′-tetramethylindodicarbocyanine (DiD), which serves as membrane probe (MP at the FIG. 2). Excitation and emission spectra of DiD do not overlap with those of Fluo-3 (C). Initially, the buffer inside and outside liposomes does not contain calcium, so Fluo-3 is not fluorescent. When calcium is added to the buffer without exposure to channel-forming agent, Fluo-3 remains calcium-free, because liposomal membrane does not allow the entry of calcium. However, after ion channels are formed in the particular liposome, the electrolytes are equilibrated between inside and outside of the liposome. Therefore, due to the presence of calcium outside, the liposomes with the channels will have high free calcium, so Fluo-3 will have intensive fluorescence.

In the flow cytometer, the appearance of channels is visible as increasing proportion of calcium-loaded liposomes compared with calcium-free liposomes. When every liposome becomes permeant to calcium, all liposomes will be presented as loaded with calcium. This condition is used for normalization and can be achieved in control experiments by the addition of calcium ionophore such as ionomycin.

If such technique is used to screen compounds for the ability to prevent the formation or function of membrane channels, we need to use the ratio of the number of liposomes in status shown at B to the total number of liposomes (both shown at A and B) as an endpoint.
A. “Intact liposomes”. Despite they are incubated in calcium-containing medium, undamaged liposomes keep intracellular calcium concentration below the threshold for binding to Fluo-3. Without ion channels, there is no fluorescence associated with Fluo-3.
B. “Liposomes with an open channel”. Oligomerized amyloid-beta binds to the lipid membrane and forms ion channels. Flow of calcium into the cell increases the concentration in the cell, which in turn shifts the calcium-binding status of Fluo-3 probe. Now, liposome emits fluorescence at 520 nm.
C. Fluorescence spectra of Fluo-3 and 1,1′-dioctadecyl-3,3,3′,3′-tetramethylindodicarbo-cyanine (DiD). Excitation and emission spectra are shown relative to the wavelengths of typical lasers used in flow cytometry (488 and 637 nm).

FIG. 4. The schematic of simplest generic fluorescence setup for the estimation of the channel size distribution of ion channels formed in artificial membranes (liposomes).

The liposomes are prepared to contain fluorescent probes of various sizes (shown as intravesicular crosshatched circles) and are cleared from extravesicular fluorescent probes. Membranes are impermeant to these probes and can contain membrane probes to identify the liposomes in the flow, because scattering usually cannot be used due to the size of unilamellar liposomes. In this example, the probes of only two sizes are shown—small and large. If liposomal membrane is impermeable, the liposome carries both probes and corresponding event in flow cytometry recording has high intensity in channels corresponding to both large and small probe. If a small channel is formed, small probe is leaking, while large remains trapped intravesicularly. Therefore, the liposome with a small channel will have fluorescence corresponding only to the large probe. Finally, if a large channel is formed, then both probes will be leaking, and liposome will have no fluorescence corresponding to either small or large probe.

FIG. 5. The example of testing set employing ten probes of different molecular weights (range 370-2000 Da), to study the distribution of molecular weight cut-offs of formed membrane channels.

The compound set was designed from the fluorescent probes available commercially to be used with a commercial flow cytometer containing four separate lasers with parallel arrangement. One of probes can be efficiently excited by two lasers, so can be detected on two separate channels. Spectra of compounds measurable in each of four channels are shown, the insert presents the distribution of molecular weights of compounds included in the set.

The sets which include compounds with molecular weights of different ranges can be built. Building the sets in the macromolecular range can dramatically benefit from availability of dextrans or other polymers with defined molecular weight, which are custom labeled with appropriate fluorescent moieties.

FIG. 6. The schematic showing arbitrary positions of liposomes containing various fluorescent labels on 2D plot representing flow cytometric data.

In this example. liposomes are made of lipid containing red fluorescent probe (such as DiD) and contain membrane-impermeant water-soluble calcium-sensitive dye which has green fluorescence (such as Fluo-3). The intensity of red fluorescence reflects the amount of membrane material in the liposome. The amount of membrane material increases in larger liposomes (elongated vesicles) and with increased number of lipid layers in the liposome (double circles). Liposomes are formed in calcium-free buffer; calcium-sensitive fluorescence probe is non-fluorescent in calcium-free solution (liposome has the same intensity of green fluorescence as one without dye) but becomes highly fluorescent after binding calcium (intensity of green fluorescence increases). Calcium level inside liposome increases when the liposome has ion channels or in the presence of ionophores.
A. Comparison of positions of unlabeled, labeled with membrane dye and filled with intravesicular dye liposomes. Unlabeled liposomes (circles with dotted membrane) do not fluoresce, however, electric noise and photobleeding are measured as low level of signal in both red and green channels. Liposomes labeled with lipophilic dye (either without water-soluble intravesicular Fluo-3 probe, or with Fluo-3 in the absence of intravesicular calcium) are labeled as circles with white filling and solid membrane. These liposomes have significant red fluorescence, but green fluorescence is at the same level as in unlabeled liposomes. Larger vesicles have more membrane-dissolved dye and have higher fluorescence. Finally, liposomes containing both membrane and intravesicular calcium-sensitive probe in the presence of calcium are labeled as circles with filling. Larger vesicles have more calcium-sensitive dye, and along with higher red fluorescence demonstrate higher green fluorescence.
B. Multilamellar liposomes have higher membrane signal. Liposomes with diameters less than 200 nm are almost exclusively unilamellar. However, in larger liposomes, some of them may be multilamellar, while liposomes with a diameter more than one micrometer would always be multilamellar. Multilamellar liposomes of the same size as unilamellar liposomes carry more membrane dye (red fluorescence is higher), but the same amount of Fluo-3 (or even slightly less)—all multilamellar liposomes will be shifted to the right in the 2D plots representing the distribution of red and green fluorescence of individual vesicles.
C. Multilamellar and unilamellar liposomes are unlikely to separate at the 2D plot. Liposomes with just membrane probe, or double-labeled liposomes without intravesicular calcium will all spread as a horizontally oriented cloud (the single-membrane ellipse with white filling). Unilamellar liposomes with intravesicular fluorescence will be represented by the elongated ellipse (with single border and filling). In log coordinates, the axis of the ellipse is directed at the 45-degree angle: due to shape, the internal volume is increasing proportionally to the surface area of membrane (linear proportion between amount of membrane and embedded dyes). Multilamellar liposomes will be looking similarly, just shifted to the right (ellipse with double border and filling). Liposomes with double lipid layer have double membrane signal, however, typically plots are made in logarithmic scale, so the separation will be impossible due to variability: the ellipses are most likely overlapping. Importantly, the aggregation of liposomes will result in the elongation of the cloud along long axis of the ellipse.

FIG. 7. Flowmetric study of phosphatidylcholine liposomes with membrane lipophilic dye DiD and embedded water-soluble 5(6)-carboxyfluorescein (CF).
A. Distribution of labeled and unlabeled liposomes. Layout of three dot plots: buffer only (darkest cloud due to the overlap with two other clouds), liposomes with membrane dye only (light grey cloud), and liposomes with both membrane and embedded water-soluble dyes (grey cloud.) Concentration of CF was 4 mM. Intensity of red-fluorescent membrane-distributed DiD is on the abscissa axis, the intensity of green-fluorescent CF—on the ordinate axis. To discriminate events in the flow, we used the threshold on the channel for red fluorescence (membrane probe amount).
B. Increase of intravesicular dye content shifts the distribution on the corresponding channel. Histograms of particles distribution for 400 nm liposomes containing DiD and various concentrations of CF: no CF or 0.2 mM, 1 mM, or 4 mM CF. Medians of the distributions corresponding to different concentrations of CF is proportional to the concentration. The gate shown at the insert (the liposomes with 4 mM CF) was used to count the histograms, and also provides an estimation of total number of events in the flow experiment.

FIG. 8. Effect of Aβ₂₅₋₃₅ on liposomes made of phosphatidylcholine is independent of calcium and can be observed in liposomes without calcium probe.

Liposomes (400 nm) were made of phosphatidylcholine with DiD and were extruded in the buffer containing 1 mM Fluo-3 (A-H) or without Fluo-3 (I, J). Addition of extravesicular calcium (A) or chelating agent EGTA (E) did not affect intensity of fluorescence. In the presence of calcium (A-D), the addition of Aβ₂₅₋₃₅ in concentrations above 10 μM slightly but reproducibly increased green signal from vesicles (B—20 μM; C—50 μM.) Addition of ionophore ionomycin (permeabilize membranes to calcium) significantly increased fluorescence (D).

The effect of Aβ₂₅₋₃₅ was the same in calcium-free medium with EGTA (F,G). As predicted, ionomycin was not affecting Fluo-3 signal in the absence of calcium (H). In liposomes extruded without Fluo-3, Aβ₂₅₋₃₅ still shifted the distribution upward (J) with the same magnitude as in liposomes containing calcium-sensitive fluorescent probe (D).

FIG. 9. Low concentrations of Aβ₂₅₋₃₅ made phosphatidylserine liposome permeant to calcium.

Liposomes (400 nm) were made of phosphatidylserine with DiD and were extruded in the buffer without (A-C) or with (D-I) 1 mM Fluo-3. Addition of extravesicular calcium (A, D) or chelating agent EGTA (G) did not affect intensity of fluorescence. Addition of 5 μM Aβ₂₅₋₃₅ increased green signal from vesicles only in the presence of both Fluo-3 and calcium (E) but did not have effect in DiD-only liposomes (B) or in calcium-free buffer (H). Addition of ionomycin did not further increase the Fluo-3 signal from vesicles with low DiD signal but additionally shifted upwards the part of the distribution with high DiD signal (F). As predicted, ionomycin did not produce any effect in DiD-only vesicles (C) and in the absence of calcium (I).
A, B, C (First row). Liposomes with membrane but without calcium-sensitive probe in the presence of calcium.
D, E, F (Second row). Liposomes with both membrane and calcium-sensitive probe in the presence of calcium.
G, H, I (Third row). Liposomes with both membrane and calcium-sensitive probe in the absence of calcium.
A, D, G (First column). Liposomes only.
B, E, H (Second column). Liposomes with added 5 μM Aβ₂₅₋₃₅.
C, F, I (Third column). Liposomes with added 5 μM Aβ₂₅₋₃₅ followed by ionomycin.

FIG. 10. The effect of Aβ₂₅₋₃₅ on liposomes made of phosphatidylserine is dose-dependent (results of a typical experiment as dot plots).

Liposomes made of phosphatidylserine and contained both DiD and Fluo-3. In a calcium-containing medium, membranes are not permeant to calcium, so there is a minimal number of liposomes with increased levels of green fluorescence (A). The addition of 2 μM Aβ₂₅₋₃₅ makes some of liposomes permeant to calcium (B). Increasing concentration of amyloid peptide increases the number of permeant liposomes (C, D).

FIG. 11. The number of phosphatidylserine liposomes permeabilized by Aβ₂₅₋₃₅ is linearly dependent on the amount of added peptide. on liposomes made of phosphatidylserine is dose-dependent (statistics).

This figure summarizes data from experiments, described at the FIG. 10. Liposomes made of phosphatidylserine and contained both DiD and Fluo-3 were treated by various concentrations of Aβ₂₅₋₃₅. For the treatment, the same stock solution of peptide in distilled water was added to the suspension of liposomes in a calcium-containing medium. The gate to count the number of permeabilized vesicles is shown at the density plot (left graph). The percent of permeabilized liposomes to total number of recorded events in each experiment is shown at the right graph.

FIG. 12. Aβ₁₋₄₂ does not permeabilize liposomes made of phosphatidylserine.

Phosphatidylserine liposomes were prepared to contain both DiD and Fluo-3 as described for previous figures. Liposomes were tested to be permeabilized by Aβ₂₅₋₃₅.
A. Control PS liposomes with calcium (60 μM). Limited number of events is registered in the area corresponding to permeabilized vesicles.
B. Effect of Aβ₁₋₄₂ (8 μM). The number of events in the area corresponding to the permeabilized vesicles does not exceed same number observed in control experiment.
C. Effect of ionomycin (50 nM, positive control). Ionomycin permeabilizes all liposomes and shifts the whole cloud of the distribution up.

FIG. 13. The technique allows to monitor the number of permeabilized liposomes as a measure of channel formation.

In this experiment, stock solution of peptides was prepared in DMSO to extend the testing to the peptide which are not soluble in water-based excipients. Peptides were added to create final concentration of 10 μM.
A. The addition of Aβ₂₅₋₃₅ to the Buffer does not create a significant number of events in areas of distribution corresponding to liposomes permeable to calcium (“Buf+Pep/DMSO”).
B. Aβ₂₅₋₃₅ dissolved initially in DMSO permeabilized liposomes similar to the same peptide dissolved in distilled water. There is no difference between the effects of the peptide that was dissolved initially in DMSO (“Lip+Pep/DMSO”) and water (not shown here).
C. Aβ₃₁₋₃₅ in the same conditions did not affect the permeability of liposomes.

FIG. 14. The schematic of fluorescence setup for selecting only unilamellar liposomes for analysis of permeabilization by membrane channels.

The separation of multilamellar from unilamellar liposomes is important because peptide-formed ion channels cannot be transferred from outer lipid layer to the internal layers, therefore multilamellar liposomes are less sensitive to permeabilization by peptides: only the space between two most peripheral layers would become equilibrated with the medium. It is important that ionophores carry ions across membranes because these molecules are both water and lipid soluble. Therefore, ionophores affect fluorescence of ion-sensitive probes in both unilamellar and multilamellar liposomes.

However, the experiments may require preparations containing relatively large liposomes—not 100-200 nm, but 400 nm—because of low intensity of fluorescence of ion-sensitive dyes. Two-fold increase of diameter results in 8-fold increase of internal volume (increasing the signal for enclosed ion-sensitive probe) and 4-fold increase of membrane surface (less membrane probe can be used, so less effect on the lipid content will be introduced).

Liposomes are created from lipids containing membrane fluorescent probe (Mem, recorded in the channel 1). Extrusion buffer contains ion-sensitive fluorescent probe (ISP, recorded in the channels 2) and volume fluorescent probe (Vol, channel 4). Also, immediately before the experiment, membrane-impermeant surface fluorescence probe (Sur, channel 3) is added.

Liposomes are identified in the flow using thresholds for membrane and volume probe. Using both signals, it is possible to select events reflecting passing liposomes of sufficient size and carrying embedded probe. Liposomes which lost volume label are excluded from the analysis (because same liposomes most likely lost ion-sensitive probe, too).

Added surface probe will bind only to an outer leaflet of membrane, therefore the ratio of membrane fluorescence to surface fluorescence allows to separate multilamellar liposomes from unilamellar ones. Unilamellar liposomes have the highest ratio of surface probe to membrane probe.

The figure simplifies the panel building at the schematics to have each fluorophore identified by using specific pair of excitation and emission wavelengths (laser-filter-detector), but clearly compensation procedure can be used where needed.
A. “Unilamellar/Channel”. Unilamellar liposomes with ion channel have high internal concentration of ion, therefore, ion-sensitive dye has high intensity of fluorescence. Membrane, volume, and surface probes are used to identify unilamellar liposome in the flow and separate it from multilamellar ones.
B. “Unilamellar/intact”. Unilamellar liposomes without ion channel have low intravesicular concentration of ion, which keeps ion-sensitive dye in non-fluorescent state. Membrane, volume, and surface probes have the same ratio to each other as in unilamellar liposomes carrying membrane channel.
C. “Multilamellar”. Multilamellar liposomes have relatively higher membrane probe content compared with unilamellar liposomes of the same size, while surface probe will be the same. Liposomes with low absolute Vol probe content are excluded, because most likely it means leakage of internal content. The remaining liposomes can be separated by calculating the ratio “Mem”/“Sur”. Liposomes with higher ration are excluded as multilamellar.

FIG. 15. The possible implementation of fluorescence setup for testing of the calcium channel formation in unilamellar liposomes with the exclusion of multilamellar liposomes.

In this specific implementation of the technique, following fluorescent probes are used: calcium-sensing probe Fluo-4; membrane probe DiD; volume probe—dextran-tetramethylrhodamine; surface probe—Pacific Blue-labeled Annexin V.

Channels are: for Pacific Blue—violet laser (405 nm)—detector with the filter covering 450 nm; For Fluo-4—blue laser (488 nm)—detector with the filter covering 520 nm; for tetramethylrhodamine—yellow laser (561 nm)—detector with the filter covering 580 nm; for DiD —red laser (637 nm)—detector with the filter covering 665 nm.
A, B, C. The same as at the FIG. 14, but channels are marked with specific wavelengths.
D. Fluorescence spectra of Pacific Blue, Fluo-4, tetramethylrhodamine, and 1,1′-dioctadecyl-3,3,3′,3′-tetramethylindodicarbo-cyanine (DiD). Excitation and emission spectra are shown relative to the wavelengths of typical lasers used in flow cytometry (vertical lines at 405, 488, 561, and 637 nm).

FIG. 16. Experimental protocols to study the effects of drugs on the ability of peptide to form membrane channel.

Depending on the sequence of adding the components into the reaction, the technique allows to distinguish effect of chemical on formation of peptide oligomers able to form ion channels, the process of incorporation of oligomer into the membrane, and the functioning of already formed channel.
A. The schematic of possible interventions to affect the function of peptide-formed membrane channel. The peptide is water soluble when freshly synthesized. After the formation of intramolecular hydrogen bonds, the peptide can form intermolecular hydrogen bonds. The resulting linear oligomer can either elongate forming protofibrils (lower path) or make an annular structure (upper path). Channels are formed by oligomeric form of peptides (several molecules of peptide, 3-4 are the most cytotoxic). Treatments can modify the formation of annular or barrel-like structures by changing the oligomerization, as well as elongation. This option is marked with the digit 1. Digit 2 marks the possibility for the treatment to affect the incorporation of channel-formed peptide into a membrane. Finally, treatments can modify the functionality of formed channel. This option is marked with digit 3. It is theoretically possible that treatments can improve the function of channel, for example, by fixing the channel in an open state, because ion channels were shown to open and close spontaneously. However, a closing of the channel will be of higher practical value.
B. Protocol to study effect of drugs on the ability to form membrane channels. Incubation of peptide solution with the drug can affect the formation of oligomers (promote aggregation into higher order polymers or prevent oligomerization). To check this possibility, the drug needs to be incubated with the peptide. The mixture is added to the prepared liposomes, and liposomes are analyzed for permeability. In this protocol, the drug is present during all three points (1-3) identified at the schematic A. Therefore, it can affect formation of oligomers, incorporation of oligomers and the functionality of the channel. To separate effectiveness at the point 1, the effect in this protocol should be compared with the effectiveness in protocols described below in Protocols C & D (effectiveness at points 2 & 3, correspondingly). Importantly, for screening compounds which are able to prevent membrane permeabilization by amyloid channels, this protocol can be applied as a first screen—if there is no effect in this screen, the compound does not prevent permeabilization by any mechanism.
C. Protocol to study effect of drugs on the incorporation of channels into membrane. Peptide oligomers are prepared in advance. Liposomes are mixed with the drug first. Then oligomers are added. Presence of drug in the solution can prevent the incorporation of oligomers in the membrane. However, the drug can affect the functionality of the already formed channel. To differentiate the option that the drug affects the incorporation vs the drug affects the functionality of channel, the results of the Protocol C should be compared with the results of Protocol D.
D. Protocol to study effect of drugs on the function of membrane channels. Peptide oligomers are prepared in advance and mixed with liposomes. This allows for membrane channel to form. The addition of drug can affect the function of already formed channels. If the channel is formed and then blocked, added ion will not be able to enter the liposome with a channel.

FIG. 17. Method of separation of liposomes permeabilized with the channels from the rest of liposomes.

This approach can be used for a purification of channels, or creating liposomal preparation enriched with liposomes carrying membrane channels.

The technique is the extension of fluorescence-activated sorting, which can be performed on commercial flow cytometers. The sample with particles (vesicles or cells) is infused into the constant flow of sheath liquid. The flow of sheath liquid is much higher than the flow of the sample, so in relatively narrow tubing cells separate from each other and become arranged in a line. Each particle passes the beam of laser individually providing the separate event which can be recorded by multiple detectors for the fluorescence and scattering where appropriate. After parameters of fluorescence of each particle are measured, and the particle reaches the outlet of the tube (due to known delay), it can be decided if particular particle should be collected in a specific collecting vessel. Usually, it is done by charging the droplet with the particle with a specific charge, which forces the droplet to change the direction in the electric field created by a pair of electrodes.

Modern flowmetry-based sorters can separate the particles into several subpopulations according to fluorescent properties. In case of liposomes, permeabilized by membrane channels, due to presence of dramatically different profile of fluorescence (such as ion-sensitive probe), the liposomes with channels can be identified in the flow (as shown at the density plot with corresponding gates). Considering that commercial sorters can separate several thousands of droplets per second, it is possible to purify liposomes in the millions. Unfortunately for protein analysis, each channel contains only several molecules of peptide, while every liposome has only one channel. Therefore, even millions of channels represent total amount of peptide below sensitivity of any possible analytical techniques. However, even now, there are applications for such preparations. First, purified liposomes with channels can be used in the testing procedures to remove excessive amounts of peptide in unrelated conformation. The analysis of inhibition of channels will not contain noise from non-permeabilized liposomes. Second, the suspensions can be used to reconstruct purified protein membrane aggregates into larger membranes for electrophysiological or similar studies. Finally, there are techniques which allow antibody generation, based on bacteriophage or similar technologies, which can generate clones of needed antibodies using single copies of separated antibody.

### DETAILED DESCRIPTION OF THE INVENTION

The invention is the method to detect membrane permeability in artificial lipid vesicles (liposomes) for ions and various compounds. The permeabilization of membrane of individual liposome is detected by measuring the fluorescence of intravesicular probe which changes when the ion or the compound of interest become able to pass the membrane of liposome (FIG. 2). The distribution of fluorescence intensities is measured by techniques well-known to those skilled in the art. One of technique is flow cytometry—the sample containing the suspension of object is passed through a narrow tube arranging the particles in a single line. Laser-excited fluorescence is measured in each individual particle, so that it is possible to study co-distribution of fluorescence intensities of multiple particles at various wavelengths. However, said distribution can be measured in other ways, such as using microscopy with digital analysis. In various embodiments of this invention, the fluorescence of individual liposomes can be increased or decreased due to their permeabilization depending on the properties of fluorescent probe used.

The permeabilization to calcium ions will be described first as an example. To detect calcium transmembrane transfer, calcium-sensitive probes are used, such as Fluo-3 or Fluo-4 which dramatically increase their fluorescence upon binding calcium. Liposomes are formed in a calcium-free medium containing calcium-sensitive probe. To make ion-sensitive dye non-fluorescent, the extrusion buffer needs to be calcium-free, that is accomplished by the addition of calcium-chelators such EGTA or EDTA. Liposomes also include membrane probe and volume probes, which have fluorescence that is independent of calcium. Membrane probe is used to identify the liposome in the flow, while volume probe is needed to confirm that there is no non-specific leakage of intravesicular content. Extravesicular probes are washed out (by dialysis, repeated centrifugation etc). An addition of calcium to the suspension of intact liposomes does not result in the increase of fluorescence, because lipid membranes are not permeant to calcium. The suspension of liposomes is subjected to flow cytometric analysis. The identification of the liposome in the flow (passing the particle through laser beam is called “an event”) is performed using fluorescence of the membrane and volume probe. The liposome, that is impermeant to the calcium, does not have fluorescence of calcium-sensitive probe, but the liposome that is permeant (for example due to the presence of membrane channel) has calcium-sensitive probe intensely fluorescing (FIG. 3). As a positive control, it is possible to use ionophore ionomycin to induce membrane permeability to calcium.

The technique can be used to study permeabilization to any ion, for which an appropriate fluorescent ion-sensitive probe can be identified. Lipid membranes are not permeant to sodium, potassium, or protons. Embodiments of this invention describe the measurement of channels translocating potassium, sodium, and protons. Calcium is added to test membrane permeability. To extend the technique to test permeability to other ions, appropriate ion-sensitive probes and ionophores need to be used (Table). Examples of extrusion and incubation buffers that are applicable to technique to detect membrane permeabilization to various ions are also shown in the Table.

| Ion | indicator | ionophore | Extrusion buffer | Incubation buffer |
|---|---|---|---|---|
| Ca²⁺ | Fluo-4 | ionomycin | EGTA | 50 mM CaCl₂ |
| Na⁺ | Sodium Green | monensin | Na⁺-free | 100 mM NaCl |
| K⁺ | PBFI | valinomycin | K⁺-free | 100 mM KCl |
| H⁺ | CF or BCECF | CCCP | pH 7.5 | pH 6.5 |

To detect non-specific membrane permeabilization to various compounds, the leakage of fluorescent compounds (such as Lucifer Yellow) themselves is studied. In this case, liposomes are formed with enclosed fluorescent compound. Intact liposomes contain the fluorescent label, while permeabilized liposome loses the compound and does not have corresponding fluorescence (FIG. 4). By using multiple enclosed compounds of various molecular weights (FIG. 5), it is possible to estimate the size of the channel in each liposome and construct the distribution of channel sizes formed under specific conditions. The use of appropriate positive controls is required. Similar to ionophores (compounds which can bind the ion and are soluble in both the medium and the membrane, so they can diffuse through undamaged membrane), channel formers such as gramicidin (antibiotic which makes membrane channels) can be utilized. Due to presence of physical openings in the membrane, volume probe with extremely high molecular weight, such as fluorescent dextran with molecular weight of 2,000,000 Da.

Also, the ability to quench or perform energy transfer can be adopted to study transmembrane transfer of various compounds. For example, permeabilization to manganese can be observed by quenching.

Our main driving force to make this invention was to study molecular mechanisms of cytotoxic effects of amyloidogenic peptides which are mediated, at least in part, by the formation of ion channels in cellular membranes. We claim that the described technique can be used for studying the effectiveness of various treatments to affect the permeability of lipid membranes to ions. We expect that this method will result in the development of high-throughput screening technique to find chemical entities that are able to prevent ion disturbances caused by amyloid-formed ion channels with overarching goal to ameliorate said disturbances and break the biochemical cascade induced by these peptides leading to neuronal death in Alzheimer's disease.

Among embodiments of this invention are the methods to select chemical entities, which are effective in the treatment of amyloid diseases. We claim that the method allows for the distinguishing treatments affecting various steps of amyloid channel formation—the creation of channel-forming units during aggregation of peptides, the incorporation of channel-forming aggregated into the membranes or affecting the function of already formed channels. The embodiments of the technique are possible to make applicable to high-throughput applications.

In another embodiment of this invention, we claim that it is possible to overcome a major limitation of studying membrane channel formation—the need of relatively large liposomes, which makes a significant ratio of vesicle being multilamellar. Considering that peptide-formed channels are formed only in the outer lipid layer, multilammelar liposomes are not an ideal study object. By adding surface probe, such as Annexin V bound to fluorescent label, liposomes can be quantified by the ratio of surface probe to membrane probe. Using only liposomes which have high ratio of surface signal to membrane signal (essentially equal amount of surface probe and membrane probe typical for unilamellar liposomes) allows to separate liposomes made of single lipid layer (unilamellar liposomes). In this way, unilammelar liposomes can be distinguished from multilamellar liposomes, and analyzed separately.

Finally, we claim that by using the extension of analytical method to identify liposomes carrying the membrane channel, the liposomes containing channels can be separated from liposomes without a channel. In this embodiment of the invention, the peptide in the form of the channel can be concentrated. The purified channels can be used not only for basic research, but also for multiple applications such as effective screening technique to identify compounds affecting amyloid membrane channel formation, and the production of macromolecules with the affinity to the channels (such as antibodies etc).

### EXAMPLES OF HOW THE INVENTION WILL BE USED

### Example 1. Measuring Effectiveness of Various Peptides to Permeabilize Membranes to Calcium

Using the invented method, we found that full-length amyloid peptide Aβ₁₋₄₂ does not create channels permeabilizing membranes to calcium (FIG. 12). In contrast, short fragment Aβ₂₅₋₃₅ efficiently permeabilize multiple liposomes (FIG. 10, in various conditions up to 10% of total number of liposomes become permeant to calcium, FIG. 11). We predict that multiple fragments of beta-amyloid, as well as other misfolding peptides and their fragments are able to permeabilize membranes by creating membrane channels. To find which peptides have channel-forming ability, it is possible to use invented technique. Most typical ion which transmembrane transport is hypothesized to be involved in neurodegeneration is calcium, so we expect that permeabilization to calcium will be attracting most of interest, at least in the beginning.

Mixture of liposomes with embedded calcium-sensitive probe is prepared. To do that, liposomes with the diameter 200 or 400 nm are extruded from phosphatidylserine containing membrane probe (i.e. DiD) in a calcium-free buffer containing calcium-sensitive probe (i.e. Fluo-4) and volume probe (i.e. dextran-tetramethylrhodamin with molecular weight 2,000,000 Da). Extravesicular probes are cleared using centrifugation. Solutions of peptides (freshly prepared or aged to allow aggregation) are added to liposomes, followed by surface probe (i.e. Annexin V bound to Pacific Blue). After short incubation, calcium is added, and the mixture is analyzed on flow cytometer. Calcium ionophore ionomycin is used as a positive control, and a vehicle for peptide serves as a negative control.

Liposomes of interest (unilamellar liposomes that retained integrity of internal content) are identified by intense fluorescence of volume probe and corresponding membrane probe. Integrity of content is controlled by the presence of volume probe. Number of lipid layers is estimated by the ratio of intensity fluorescence of membrane probe to surface probe. Unilamellar liposomes have the lowest ratio. In identified liposomes, the concentration of calcium is estimated. Liposomes without channels have low calcium, and corresponding low fluorescent signal of Fluo-4. Liposomes with channels have high calcium and intense fluorescence of Fluo-4. The ratio of the number of liposomes with channels to total number of liposomes (or to the number of liposomes without channels) is the endpoint of test. Peptides which statistically significantly increase the ratio of permeabilized liposomes are considered channel-forming.

### Example 2. Measuring Permeabilization by Various Peptides to Various Ions

Based on previous experimental data, it is reasonable to expect that amyloid membrane channels formed by various peptides are non-selective and can pass various ions (sodium, potassium, or protons).

Mixture of liposomes with embedded ion-sensitive probes is prepared. To do that, liposomes with the diameter 200 or 400 nm are extruded from phosphatidylserine containing membrane probe (i.e. DiD) in a appropriate buffer containing one or several ion-sensitive probe (see the Table in the detailed description of the invention) and volume probe (i.e. dextran-tetramethylrhodamin with molecular weight 2,000,000 Da). Extravesicular probes are cleared using centrifugation. Solutions of peptides (freshly prepared or aged to allow aggregation) are added to liposomes, followed by surface probe (i.e. Annexin V bound to Pacific Blue). After short incubation, test ions are added, and the mixture is analyzed on flow cytometer. Appropriate ionophores are used as a positive control for permeabilization to a specific ion, and a vehicle for peptide serves as a negative control.

### Example 3. Screening Chemical Compounds for the Ability to Prevent Membrane Permeabilization Through the Formation of Peptide Channels

The method for screening chemical entities for an ability to prevent membrane permeabilization induced by misfolding peptides through membrane channel formation will be used to find drug candidates to treat neurodegenerative diseases. For example, chemical entities able to prevent channel functioning induced by amyloid peptides can be effective in the prevention or in the treatment of Alzheimer's disease.

A suspension of liposomes with embedded ion-sensitive probes is prepared. To do that, liposomes with the diameter 200 or 400 nm are extruded from phosphatidylserine containing membrane probe (i.e. DiD) in a appropriate buffer containing one or several ion-sensitive probe and volume probe. Extravesicular probes are cleared using centrifugation. Solutions of peptide (freshly prepared or aged to allow aggregation) are added to liposomes, followed by surface probe (i.e. Annexin V bound to Pacific Blue). After short incubation, test ions are added, and the mixture is analyzed on flow cytometer. Appropriate ionophores are used as a positive control for permeabilization to a specific ion, and a vehicle for peptide serves as a negative control.

Chemical entity that significantly decrease the ratio of permeabilized liposomes to the total number of liposomes is considered effective against channel-mediated permeability of membranes.

Tested drug is added to the test system at various stages to dissect which step of channel formation and function is affected by the drug. First, the drug is mixed and pre-incubated with channel-forming peptide. Considering that the drug is present at all stages—formation of channel-forming units in the solution, incorporation of channels into the membrane, and when the membrane channel transports ion, this timing can be applied as a first screen—if there is no effect in this screen, the compound does not prevent permeabilization by any mechanism.

Alternatively, the drug is added and pre-incubated with liposomes. The channel-forming peptide is added in the presence of the drug. In this case, the drug can prevent the incorporation of the channel and the permeability of formed channel. The comparison with the previous timeline, will allow for an identification of drug effect on aggregation of peptide into channel-forming units.

Finally, channel-forming peptide can be added to liposomes first. If drug is added immediately before adding test ion, the drug can only affect the functionality of the formed channel. By comparing three sequences, it is possible to dissect the mechanism of anti-permeabilization effect of the drug. As it was mentioned, for the purposed of high-throughput screening, it will be logical to apply the first sequence (drug is co-incubated with the peptide), which allows to identify drugs which are not effective against membrane permeabilization by misfolding peptide by any mechanism.

### Example 4. Purification of Channels

The embodiment of the technique which includes flow sorting allows to separate liposomes which have ion channels from ones without channel. Essentially, it is functional purification of the protein in the form of channel. Formed channels are relatively stable, therefore, collected suspension of purified channels incorporated into liposomes can be stored at least for a limited time, and even transported to those who can use them for their own applications. Purified liposomes with channels can serve as a study object. They also can be used as a test object in screening applications if non-purified pool of liposomes contains too many other objects. The excessive number of other objects can be detrimental, for example, in detecting rare events or where total non-specific absorption on lipid could be an issue.

### Example 5. Creation of Antibodies Selectively Affecting Channels

Purified channels incorporated into the liposomes can be used in various applications to develop macromolecules with affinity to channels (such as antibodies). The amount of purified peptide would be most likely not sufficient for typical immunization protocol, because each liposome contains only a single channel (essentially a single macromolecular complex to be targeted by antibody). However, those who are skilled in arts, can apply alternative techniques which can be effective with negligibly small amount of available antigen, such as phage-based technologies to generate affine molecules.

Generated macromolecules with a specific affinity to peptides in a channel form can be a therapeutic in the treatment of degenerative diseases which are caused by said peptides. Also, the antibodies can be a research and/or diagnostic tool to label this pathophysiologically relevant marker in biological samples.

### Example 6. Quantitative Estimation of Channel-Forming Units in a Sample

After Aβ₂₅₋₃₅ is added to the liposomal preparation, the effects of the peptide develop within the first minute. The incubation of liposomes with the peptide for up to one hour does not change the number of permeabilized liposomes. That means that the interaction of the peptide with the membrane occurs quickly and once inserted into the membrane, a peptide aggregate that already formed a channel is not able to affect other vesicles. It can be concluded that the solutions contain some amount of peptide which is ready to incorporate into membranes and form channels. We named such peptide aggregates “channel-forming units”.

There is a linear relationship between the number of added units (concentration of added peptide) and the number of liposomes permeabilized by the channels. Therefore, each permeabilized liposome carries a single channel, so the number of permeabilized liposomes reflects the number of formed channels and can be used as a test system.

### Example 7. Testing the Ability of Sample to Produce Peptide Channels from Long Peptides

Our core hypothesis of the etiology and pathogenesis of Alzheimer's disease (which we believe is relevant to other degenerative diseases) is that proteolytic enzymes digest long peptides into shorter fragments which are able to form membrane channels. Permeabilization of cellular membranes by channels initiates biochemical processes leading to cell death. In one of embodiments of this invention, the process of membrane channel formation from fragments produced by proteases from longer peptides is monitored.

A suspension of liposomes with embedded ion-sensitive probes is prepared. To do that, liposomes with the diameter 200 or 400 nm are extruded from phosphatidylserine containing membrane probe (i.e. DiD) in an appropriate buffer containing one or several ion-sensitive probe and volume probe. Extravesicular probes are cleared using centrifugation.

Proteases (pure enzymes, their mixtures, or biological samples with proteolytic activity) are added and mixed with the solutions of proteins. During incubation of resulting sample, the aliquots are taken over time. The aliquots are added to the liposomal preparations and the percentage of permeabilized liposomes is estimated. Considering that invented method provides the measurement of the number of channel-forming units in the sample, it is possible to observe the number of channel-forming units produced by fragments produced by proteases from long peptide.

Drugs, which are tested for the ability to modify proteolytic activity, can be added together with proteases to the long peptide. Drug-induced change of the number of permeabilized liposomes produced by products of the proteolytic digestion can be used to screen chemical entities with anti-degenerative properties.

1. A method of quantifying induced changes of membrane permeability to various substances comprising the use of preparations of liposomes containing enclosed fluorescent probes which are measured by flow cytometry.

2. The method of claim 1, wherein said liposomes also contain a set of dyes (in various combinations): membrane dye to identify the liposome in the flow; volume intravesicular dye to identify the integrity of internal volume of specific liposome; surface fluorescent probe to distinguish unilamellar liposomes from multilamellar liposomes.

3. The method of claim 1, wherein permeability to ions is measured and said liposomes contain ion-sensitive fluorescent probes.

4. The method of claim 1, wherein liposomes are prepared to contain initially one or more fluorescent probes with different molecular weights and/or spatial properties, such a globular vs rod-like, stiff vs flexible structures) with the permeabilization identified by leaking of said probes, so the distribution of channel sizes can be constructed from measuring the leakage of particular probes from multiple individual liposomes.

5. The method of claim 1, wherein permeability of membranes is changed by membrane channels formed by peptides (including but not limited to full-lengths peptides, their fragments, mutations, and derivatives: beta-amyloid, alpha-synuclein, tau-protein, amylin, huntingtin, superoxide dismutase, TDP-43).

6. The method of claim 5 to detect membrane channels made by misfolding peptides.

7. The method of claim 5 to detect membrane channels made by peptides implicated in the development of neurodegenerative diseases.

8. The method of claim 1, wherein the number of channel-forming units in the biological samples are estimated.

9. The method of claim 8, wherein the effect of treatments (chemical entities, biologically active molecules, and/or physical conditions) on peptide-induced changes of membrane permeability is estimated.

10. The method of claim 9, wherein screening of chemical libraries is performed to find chemical entities able to prevent said induced membrane permeability.

11. The method of claim 9, wherein the treatment is the proteolytic enzyme digesting full-length peptide and producing channel-forming fragments.

12. The method of claim 11, wherein the treatment is the mixture of said proteolytic enzyme with a chemical entities or biologics which have a potential to inhibit said proteolytic peptide.

13. The method of claim 12, wherein said method is applied to screen a library of chemical entities and biologics to find chemical entities or biologics able to prevent membrane permeability induced by peptide fragments produced by proteolytic enzymes digesting full-length peptide.

14. The method of claim 13, wherein such chemical entities are intended to treat degenerative diseases (including but not limited to Alzheimer's disease, Parkinson's disease, Lou Gehrig's disease (amyotrophic lateral sclerosis), Huntington's disease, diabetes, diseases caused by prions, Down syndrome).

15. The method of claim 5, wherein the step of channel formation affected by said treatment is determined by comparison of results obtained in experiments, when the drug, liposomes, and peptide are mixed with each other in different order: channel blockers work when added at any time, inhibitors of aggregation need to be mixed with the peptide and allowed to affect the aggregation, and inhibitors of incorporation into the membrane should be added to the liposomes before the addition of peptide.

16. The method of claim 5, wherein after estimating the presence, absence, or the size of incorporated channel in each liposome, the liposomes are collected according to this presence, absence, or size of the channel.

17. The method of claim 16, wherein the separation of liposome is performed using flow cytometer with sorting capability.

18. The method of claim 16, wherein separated liposomes are collected and used to produce products with the affinity to the channels (such as antibodies).

19. The method of claim 18, wherein said products with the affinity to the channels (such as antibody) are able to inactivate/prevent/ameliorate induced membrane permeability.

20. The method of claim 19, wherein said products with the affinity to the channels (such as antibody) are intended to treat diseases caused by induced membrane permeability.
